(19) United States
(12) Patent Application Publication
(10) Pub. No.: US 2016/0375112 A1
(43) Pub. Date: Dec. 29, 2016

(54) Reducing Adverse Side Effects of a Compound by a Neurotoxin

(71) Applicant: TOXCURE, LLC, Demarest, NJ (US)

(72) Inventors: Christopher Shaari, Demarest, NJ (US); Doris Day, New York, NY (US)

(21) Appl. No.: 14/901,839

(22) PCT Filed: Jul. 3, 2014
(86) PCT No.: PCT/US14/45403

§ 371 Date: Dec. 29, 2015

Related U.S. Application Data
(60) Provisional application No. 61/842,494, filed on Jul. 3, 2013.

(51) Int. Cl. A61K 38/48; A61K 9/00; A61K 31/4745

(52) U.S. Cl. CPC A61K 38/4893; A61K 31/4745; A61K 9/0014; C12Y 304/24069

(57) ABSTRACT

The present invention relates to methods and compositions for treating various side effects associated with the administration of one or more therapeutic compounds. In particular embodiments, the present invention relates to methods of reducing one or more adverse side effects associated with one or more therapeutic compounds by administering a neurotoxin, such as botulinum toxin, in combination with the one or more therapeutic compounds.

### CROSS-REFERENCE TO RELATED APPLICATION

The present application claims the benefit of priority of U.S. Application No. 61/842,494, filed Jul. 3, 2013, which is incorporated by reference herein in its entirety.

### FIELD OF THE INVENTION

The present invention relates to methods for treating various side effects associated with the administration of one or more therapeutic compounds. In particular embodiments, the present invention relates to methods of reducing adverse side effects associated with one or more therapeutic compounds by administering a neurotoxin, such as botulinum toxin, in combination with the one or more therapeutic compounds.

### BACKGROUND OF THE INVENTION

Often, adverse side effects are associated with the administration of a therapeutic compound, such as chemotherapeutic agents or topical steroids. Thus, in addition to and/or as a result of the efficacious actions of the therapeutic compound, such as selectively targeting skin cancer cells for cell death, unwanted side effects, such as redness, blistering, and/or pain at the site of application of the therapeutic compound, can sometimes occur.

Various therapeutic compounds, such as chemotherapeutic compounds, are discussed in more detail below.

Alkylating agents directly damage DNA to prevent the cancer cell from reproducing. As a class of drugs, these agents are not phase-specific; in other words, they work in all phases of the cell cycle. Alkylating agents are used to treat many different cancers, including acute and chronic leukemia, lymphoma, Hodgkin disease, multiple myeloma, sarcoma, as well as cancers of the lung, breast, and ovary. Because these drugs damage DNA, they can cause long-term damage to the bone marrow. In a few rare cases, this can eventually lead to acute leukemia. The risk of leukemia from alkylating agents is “dose-dependent,” meaning that the risk is small with lower doses, but goes up as the total amount of drug used gets higher. The risk of leukemia after alkylating agents is highest 5 to 10 years after treatment. There are many different alkylating agents, including: nitrogen mustards, such as mechlorethamine (nitrogen mustard), chlorambucil, cyclophosphamide (Cytoxan®), ifosfamide, and melphalan; nitrosoureas, such as streptozocin, carmustine (BCNU), and lomustine; alkyl sulfonates, which include busulfan; triazines, such as dacarbazine (DTIC), and temozolomide (Temodar®); and ethylenimines such as thiotepa and altretamine (hexamethylmelamine). The platinum drugs (cisplatin, carboplatin, and oxalaplatin) are sometimes grouped with alkylating agents because they kill cells in a similar way. These drugs are less likely than the alkylating agents to cause leukemia.

Antimetabolites are a class of drugs that interfere with DNA and RNA growth by substituting for the normal building blocks of RNA and DNA. These agents damage cells during the S phase. They are commonly used to treat leukemias, tumors of the breast, ovary, and the intestinal tract, as well as other cancers. Examples of antimetabolites include 5-fluorouracil (5-FU), capecitabine (Xeloda®), 6-mercaptopurine (6-MP), methotrexate, gemcitabine (Gemzar®), cytarabine (Ara-C®), fludarabine, and pemetrexed (Alimta®).

Anthracyclines are anti-tumor antibiotics that interfere with enzymes involved in DNA replication. These agents work in all phases of the cell cycle. Thus, they are widely used for a variety of cancers. A major consideration when giving these drugs is that they can permanently damage the heart if given in high doses. For this reason, lifetime dose limits are often placed on these drugs. Examples of anthracyclines include daunorubicin, doxorubicin (Adriamycin®), epirubicin, and idarubicin. Other anti-tumor antibiotics include the drugs actinomycin-D, bleomycin, and mitomycin-C.

Mitoxantrone is an anti-tumor antibiotic that is similar to doxorubicin in many ways, including the potential for damaging the heart. This drug also acts as a topoisomerase II inhibitor, and can lead to treatment-related leukemia. Mitoxantrone is used to treat prostate cancer, breast cancer, lymphoma, and leukemia.

Topoisomerase inhibitors interfere with enzymes called topoisomerases, which help separate the strands of DNA so they can be copied. They are used to treat certain leukemias, as well as lung, ovarian, gastrointestinal, and other cancers. Examples of topoisomerase I inhibitors include topotecan and irinotecan (CPT-11). Examples of topoisomerase II inhibitors include etoposide (VP-16) and teniposide. Treatment with topoisomerase II inhibitors increases the risk of a second cancer—acute myelogenous leukemia. Secondary leukemia can be seen as early as 2-3 years after the drug is given.

Mitotic inhibitors are often plant alkaloids and other compounds derived from natural products. They can stop mitosis or inhibit enzymes from making proteins needed for cell reproduction. These drugs work during the M phase of the cell cycle, but can damage cells in all phases. They are used to treat many different types of cancer including breast, lung, myelomas, lymphomas, and leukemias. These drugs are known for their potential to cause peripheral nerve damage, which can be a dose-limiting side effect. Examples of mitotic inhibitors include: the taxanes, such as paclitaxel (Taxol®) and docetaxel (Taxotere®); epothilones, which include ixabepilone (Ixempra®); the vinca alkaloids, such as vinblastine (Velban®), vincristine (Oncovin®), and vinorelbine (Navelbine®); and estramustine (Emcyt®).

Steroids are natural hormones and hormone-like drugs that are useful in treating some types of cancer (lymphoma, leukemias, and multiple myeloma), as well as other illnesses. When these drugs are used to kill cancer cells or slow their growth, they are considered chemotherapy drugs. Corticosteroids are commonly used as anti-emetics to help prevent nausea and vomiting caused by chemotherapy, too. They are also used before chemotherapy to help prevent severe allergic reactions (hypersensitivity reactions). Examples include prednisone, methylprednisolone (Solumedrol®) and dexamethasone (Decadron®).

Some chemotherapy drugs act in slightly different ways and do not fit well into any of the other categories. Examples include drugs such as L-asparaginase, which is an enzyme, and the proteosome inhibitor bortezomib (Velcade®).

While chemotherapy drugs take advantage of the fact that cancer cells divide rapidly, other drugs target different properties that set cancer cells apart from normal cells. They often have less serious side effects than those commonly caused by chemotherapy drugs because they are targeted to work mainly on cancer cells, not normal, healthy cells. Many are used along with chemotherapy.

As researchers have come to learn more about the inner workings of cancer cells, they have begun to create new drugs that attack cancer cells more specifically than traditional chemotherapy drugs can. Most attack cells with mutant versions of certain genes, or cells that express too many copies of a particular gene. These drugs can be used as part of primary treatment or after treatment to maintain remission or decrease the chance of recurrence. Only a handful of these drugs are available at this time. Examples include imatinib (Gleevec®), gefitinib (Iressa®), erlotinib (Tarceva®), sunitinib (Sutent®) and bortezomib (Velcade®).

Differentiating agents act on the cancer cells to make them mature into normal cells. Examples include the retinoids, tretinoin (ATRA or Atralin®) and bexarotene (Targretin®), as well as arsenic trioxide (Arsenox®).

Hormone therapy includes the use of sex hormones, or hormone-like drugs, that alter the action or production of female or male hormones. They are used to slow the growth of breast, prostate, and endometrial (uterine) cancers, which normally grow in response to natural hormones in the body. These cancer treatment hormones do not work in the same ways as standard chemotherapy drugs, but rather by preventing the cancer cell from using the hormone it needs to grow, or by preventing the body from making the hormones. Examples include: the anti-estrogens—fulvestrant (Faslodex®), tamoxifen, and toremifene (Fareston®); aromatase inhibitors—anastrozole (Arimidex®), exemestane (Aromasin®), and letrozole (Femara®); progestins megestrol acetate (Megace®); estrogens; anti-androgens—bicalutamide (Casodex®), flutamide (Eulexin®), and nilutamide (Nilandron®); and LHRH agonists—leuprolide (Lupron®) and goserelin (Zoladex®).

Some drugs are given to people with cancer to stimulate their natural immune systems to more effectively recognize and attack cancer cells. These drugs offer a unique method of treatment, and are often considered to be separate from chemotherapy. Compared to other forms of cancer treatment such as surgery, radiation therapy, or chemotherapy, immunotherapy is still relatively new. There are different types of immunotherapy. Active immunotherapies stimulate the body's own immune system to fight the disease. Passive immunotherapies do not rely on the body to attack the disease; instead, they use immune system components (such as antibodies) created outside of the body. Types of immunotherapies include: monoclonal antibody therapy (passive immunotherapies)—rituximab (Rituxan®) and alemtuzumab (Campath®); non-specific immunotherapies and adjuvants (other substances or cells that boost the immune response)—BCG, interleukin-2 (IL-2), and interferon-alpha; immunomodulating drugs—thalidomide, lenalidomide (Revlimid®), and pomalidomide; cancer vaccines (active specific immunotherapies)—several vaccines are being studied, but the only FDA-approved vaccine to treat cancer thus far is Sipuleucel-T (Provenge®) (American Cancer Society, Inc. website, 2014).

The anaerobic, gram positive bacterium, Clostridium botulinum, produces a potent polypeptide neurotoxin, referred to as botulinum toxin. To date, seven immunologically distinct botulinum neurotoxins have been characterized: serotypes A, B, C₁, D, E, F, and G. Of these, botulinum toxin serotype A is recognized as one of the most lethal naturally occurring agents.

It is thought that botulinum toxins bind with high affinity to cholinergic motor neurons, are transferred into the neuron and effectuate blockade of the presynaptic release of acetylcholine. All of the botulinum toxin serotypes are purported to inhibit release of acetylcholine at the neuromuscular junction. They do so by affecting different neurosecretory proteins and/or cleaving these proteins at different sites. For example, botulinum toxin serotype A is a zinc endopeptidase which can specifically hydrolyze a peptide linkage of the intracellular, vesicle associated protein SNAP-25. Botulinum toxin serotype E also cleaves the 25 kiloDalton (kD) synaptosomal associated protein (SNAP-25); however, serotype E binds to a different amino acid sequence within SNAP-25. It is believed that differences in the site of inhibition are responsible for the relative potency and/or duration of action of the various botulinum toxin serotypes.

Currently, botulinum toxins have been used in clinical settings for the treatment of neuromuscular disorders characterized by hyperactive skeletal muscles. Botulinum toxin serotype A was approved in 1989 by the U.S. Food and Drug Administration (FDA) for the treatment of blepharospasm, strabismus, and hemifacial spasm in patients over the age of twelve. In 2000, the FDA approved commercial preparations of botulinum toxin serotype A and serotype B for the treatment of cervical dystonia, and in 2002, the FDA approved botulinum toxin serotype A for the cosmetic treatment of certain hyperkinetic (glabellar) facial wrinkles. In 2004, the FDA approved botulinum toxin for the treatment of hyperhidrosis. Non-FDA approved uses include treatment of hemifacial spasm, spasmodic torticollis, oromandibular dystonia, spasmodic dysphonia and other dystonias, tremor, myofascial pain, temporomandibular joint dysfunction, migraine, and spasticity.

Clinical effects of peripheral intramuscular botulinum toxin serotype A are usually seen within 24-48 hours of injection and sometimes within a few hours. When used to induce muscle paralysis, symptomatic relief from a single intramuscular injection of botulinum toxin serotype A can last approximately three months; however, under certain circumstances, effects have been known to last for several years.

Despite the apparent difference in serotype binding, it is thought that the mechanism of botulinum activity is similar and involves at least three steps. First, the toxin binds to the presynaptic membrane of a target cell. Second, the toxin enters the plasma membrane of the effected cell wherein an endosome is formed. The toxin is then translocated through the endosomal membrane into the cytosol. Third, the botulinum toxin appears to reduce a SNAP disulfide bond resulting in disruption in zinc (Zn++) endopeptidase activity, which selectively cleaves proteins important for recognition and docking of neurotransmitter-containing vesicles with the cytoplasmic surface of the plasma membrane, and fusion of the vesicles with the plasma membrane. Botulinum toxin serotypes B, D, F, and G cause degradation of synaptobrevin (also called vesicle-associated membrane protein (VAMP)), a synaptosomal membrane protein. Most of the VAMP present at the cytosolic surface of the synaptic vesicle is removed as a result of any one of these cleavage events. Each toxin specifically cleaves a different bond.

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin serotype A complex can be produced by Clostridial bacterium as 900 kD, 500 kD, and 300 kD forms. Botulinum toxin serotypes 13 and C₁ are apparently produced as only a 500 kD complex. Botulinum toxin serotype D is produced as both 300 kD and 500 kD complexes. Finally, botulinum toxin serotypes E and F are produced as only approximately 300 kD complexes. The complexes (e.g., molecular weight greater than about 150 kD) are believed to contain a non-toxin hemagglutinin protein and a non-toxin and non-toxic nonhemagglutinin protein. These two non-toxin proteins (which along with the botulinum toxin molecule can comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex. The toxin complexes can be dissociated into toxin protein and hemagglutinin proteins by treating the complex with red blood cells at pH 7.3. The toxin protein has a marked instability upon removal of the hemagglutinin protein.

All the botulinum toxin serotypes are made by Clostridium botulinum bacteria as inactive single chain proteins which must be cleaved or nicked by proteases to become neuroactive. The bacterial strains that make botulinum toxin serotypes A and G possess endogenous proteases, and serotypes A and G can therefore be recovered from bacterial cultures in predominantly their active form. By contrast, botulinum toxin serotypes C₁, D, and E are synthesized by nonproteolytic strains and are therefore typically unactivated when recovered from culture. Botulinum toxin serotypes B and F are produced by both proteolytic and nonproteolytic strains and therefore can be recovered in either the active or inactive form. However, even the proteolytic strains that produce, for example, botulinum toxin serotype B only cleave a portion of the toxin produced. The exact proportion of nicked to unnicked molecules depends on the length of incubation and the temperature of the culture. Therefore, a certain percentage of any preparation of, for example, the botulinum toxin serotype B toxin is likely to be inactive, possibly accounting for a lower potency of botulinum toxin serotype B as compared to botulinum toxin serotype A. The presence of inactive botulinum toxin molecules in a clinical preparation will contribute to the overall protein load of the preparation, which has been linked to increased antigenicity, without contributing to its clinical efficacy.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP, and glutamate.

High quality crystalline botulinum toxin serotype A can be produced from the Hall A strain of Clostridium botulinum with characteristics of 3×10⁷ U/mg, an A₂₆₀/A₂₇₈ of less than 0.60, and a distinct pattern of banding on gel electrophoresis. The known Shantz process can be used to obtain crystalline botulinum toxin serotype A, as set forth in Shantz, E. J., et al, Properties and use of Botulinum toxin and Other Microbial Neurotoxins in Medicine, Microbiol. Rev. 56: 80-99 (1992). Generally, the botulinum toxin serotype A complex can be isolated and purified from an anaerobic fermentation by cultivating Clostridium botulinum serotype A in a suitable medium. Raw toxin can be harvested by precipitation with sulfuric acid and concentrated by ultramicrofiltration. Purification can be carried out by dissolving the acid precipitate in calcium chloride. The toxin can then be precipitated with cold ethanol. The precipitate can be dissolved in sodium phosphate buffer and centrifuged. Upon drying there can then be obtained approximately 900 kD crystalline botulinum toxin serotype A complex with a specific potency of 3×10⁷ LD₅₀ U/mg or greater. This known process can also be used, upon separation out of the non-toxin proteins, to obtain pure botulinum toxins, such as for example: purified botulinum toxin serotype A with an approximately 150 kD molecular weight with a specific potency of 1-2×10⁸ LD₅₀ U/mg or greater; purified botulinum toxin serotype B with an approximately 156 kD molecular weight with a specific potency of 1-2×10⁸ LD₅₀ U/mg or greater, and; purified botulinum toxin serotype F with an approximately 155 kD molecular weight with a specific potency of 1-2×10⁷ LD₅₀ U/mg or greater.

Already prepared and purified botulinum toxins and toxin complexes suitable for preparing pharmaceutical formulations can be obtained from List Biological Laboratories, Inc., Campbell, Calif.; the Centre for Applied Microbiology and Research, Porton Down, U.K.; Wako (Osaka, Japan), as well as from Sigma Chemicals of St Louis, Mo.

The pattern of toxin spread within a muscle has been demonstrated to be related to concentration, volume, and location of injection site.

It is noted that in this disclosure and particularly in the claims, terms such as “comprises,” “comprised,” “comprising,” and the like can have the meaning attributed to it in U.S. patent law; e.g., they can mean “includes,” “included,” “including,” and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Citation or identification of any document in this application is not an admission that such document is available as prior art to the present invention.

### SUMMARY OF THE INVENTION

The instant invention relates to methods and compositions for the reduction of one or more side effects associated with a therapeutic compound. In certain embodiments, the inventive methods provide for the reduction of one or more adverse side effects associated with a therapeutic compound, wherein a neurotoxin is administered to a subject in combination with a therapeutic compound, and wherein the adverse side effects typically associated with the therapeutic compound are reduced (e.g., are mild or moderate instead of severe) or do not occur.

In certain embodiments, the invention relates to a method of reducing one or more side effects associated with a therapeutic compound in a subject, comprising administering to the subject a therapeutically effective amount of neurotoxin in combination with the therapeutic compound, wherein the therapeutically effective amount of neurotoxin reduces one or more side effects of the therapeutic compound.

In some embodiments, the invention relates to a method of reducing one or more side effects associated with one or more therapeutic compounds in a subject, comprising administering to the subject a therapeutically effective amount of neurotoxin in combination with the one or more therapeutic compounds, wherein the therapeutically effective amount of neurotoxin reduces one or more side effects associated with one or more of the therapeutic compounds.

In other embodiments, the invention relates to a method of reducing one or more side effects associated with a combination of two or more therapeutic compounds in a subject, comprising administering to the subject a therapeutically effective amount of neurotoxin in combination with the two or more therapeutic compounds, wherein the therapeutically effective amount of neurotoxin reduces one or more side effects associated with the combination of two or more therapeutic compounds.

In some embodiments, the neurotoxin is administered before the therapeutic compound is administered. In other embodiments, the neurotoxin is administered at the same time as the therapeutic compound. In yet other embodiments, the neurotoxin is administered after the therapeutic compound.

In further embodiments, the therapeutic compound is administered topically.

In some embodiments, the therapeutic compound is selected from the group consisting of: an alkylating agent, an antimetabolite, an anthracycline, mitoxantrone, a topoisomerase inhibitor, a mitotic inhibitor, a steroid, a differentiation agent, a hormone, and an immunotherapy agent.

In a particular embodiment, the therapeutic compound is imiquimod. In further embodiments, the imiquimod is administered topically. In a particular embodiment, methods are provided for the reduction of one or more unwanted side effects associated with topical administration of imiquimod (e.g., ALDARA® cream, Medicis Pharmaceutical Corporation, Scottsdale, Ariz.), wherein botulinum toxin is administered in combination with topically administered imiquimod (e.g., ALDARA® cream) to a subject in need of treatment with imiquimod, and wherein adverse side effects associated with the topical administration of imiquimod (e.g., ALDARA® cream), such as pain, blistering, redness, and/or sensitivity at the site of application of the imiquimod (e.g., ALDARA® cream), are mild.

In some embodiments, one or more side effects associated with a therapeutic compound that are reduced by the methods described herein are adverse side effects at or near the site of administration of the therapeutic compound wherein the adverse side effects are selected from the group consisting of: pain, erythema, soreness, swelling, blistering, and sensitivity. In a particular embodiment, these adverse side effects are associated with the therapeutic compound, imiquimod. In further embodiments, the imiquimod is administered to the subject topically (e.g., ALDARA® cream). In certain embodiments, the site of topical administration of imiquimod (e.g., ALDARA® cream) is to a neoplasm, an actinic keratosis, or a genital wart. In some embodiments, the neurotoxin is botulinum toxin and is applied to the non-cancerous area around the neoplasm. In further embodiments, the neoplasm is a basal cell carcinoma. In certain embodiments, the basal cell carcinoma is superficial or nodular.

In some embodiments, the side effects of the therapeutic compound (e.g., imiquimod) are reduced such that they are mild or do not occur.

In certain embodiments, the neurotoxin is botulinum toxin. In a further embodiment, the dose of botulinum toxin does not exceed 500 units per application. In one embodiment, the dose of botulinum toxin is between about 0.01 to about 100 units per application. In another embodiment, the dose of botulinum toxin is between about 1 unit to about 50 units per application. In some embodiments, the botulinum toxin is botulinum toxin type A. In other embodiments, the botulinum toxin is botulinum toxin type B.

In yet another embodiment, the neurotoxin (e.g., botulinum toxin) is applied topically, by inhalation, or by injection. In one embodiment, the neurotoxin is botulinum toxin and is applied by injection.

The neurotoxin (e.g., botulinum neurotoxin) may be administered via a single injection or multiple injections. The neurotoxin (e.g., botulinum neurotoxin) may also be administered by aerosol.

The neurotoxin (e.g., botulinum neurotoxin) may be administered at the same site of administration as the therapeutic compound, in the vicinity of the therapeutic compound, or at a site distant to the therapeutic compound. Both the therapeutic compound and the neurotoxin (e.g., botulinum neurotoxin) may be administered by any suitable means, including administration topically, by injection, by inhalation, or any combination thereof. For example, in some embodiments, the neurotoxin is administered by injection and the therapeutic compound is administered topically. In other embodiments, the neurotoxin is administered by injection and the therapeutic compound is administered orally. In certain embodiments, both the therapeutic compound and neurotoxin are administered by injection. In other embodiments, both the therapeutic compound and neurotoxin are administered topically.

In still another embodiment of the invention, the neurotoxin (e.g., botulinum neurotoxin) may be injected into local, regional, or distant lymphoid tissue, which can be done with visual (e.g., eye or scope) or radiographic guidance such as a CAT scan or ultrasound guidance.

In certain embodiments, the neurotoxin (e.g., botulinum neurotoxin) may be applied to, but not limited to the following sites: regional muscles (including at the microscopic level) area surrounding regional lymphoid tissues; the regional nodal basins; the thymus; spleen; and bone marrow or other hematopoietic sites.

In one embodiment, the neurotoxin (e.g., botulinum neurotoxin) denervates muscle tissue surrounding the injection site and/or minimizes and/or stops lymphatic flow in the region of the injection site of the neurotoxin.

In another embodiment, the neurotoxin is botulinum toxin and weakens contraction of muscle fibers in the region of the injection site of the botulinum neurotoxin.

### DETAILED DESCRIPTION OF THE INVENTION

Described herein are methods and compositions for the reduction of one or more unwanted side effects associated with a therapeutic compound. In certain embodiments, the inventive methods provide for the reduction of one or more adverse side effects associated with a therapeutic compound, wherein a neurotoxin, such as botulinum toxin, is administered to a subject in combination with a therapeutic compound, such as imiquimod (e.g., ALDARA® cream), and wherein the adverse side effects typically associated with the therapeutic compound (such as, in the case of imiquimod administered via ALDARA® cream, pain, blistering, redness, and/or sensitivity at the site of application of the ALDARA® cream) are reduced (e.g., are mild) or do not occur.

In particular embodiments, the neurotoxin is botulinum toxin, and it is administered in conjunction with a therapeutic compound to reduce, ameliorate, prevent, or eliminate an unwanted or unpleasant side effect associated with the therapeutic compound. The therapeutic compound may be used in the treatment of any disease or condition. The administration of the botulinum toxin can occur before, at the same time as, or subsequent to the administration of the therapeutic compound for which a reduction in adverse side effects is desired.

For topically administered therapeutic compounds, the neurotoxin, such as botulinum toxin, can be administered near or at the same site as the administration site of the therapeutic compound. For example, in one embodiment, botulinum toxin is administered to the non-cancerous area around a neoplasm, such as by injection into non-cancerous cells around a nodular basal cell carcinoma, prior to administration of a therapeutic agent for the treatment of the neoplasm, such as imiquimod (e.g., ALDARA® cream) that is applied directly to and around the nodular basal cell carcinoma, that is, to the cancer and to the normal surrounding tissue.

In certain embodiments, the methods described herein enable the use of lower doses of the therapeutic agent and/or reduce the time period of application of the therapeutic agent.

Topical imiquimod (e.g., ALDARA® cream) therapy is used in the treatment of a number of conditions, in spite of its adverse side effects, which can include soreness, pain, blistering, redness, and/or sensitivity at the site of application of the imiquimod (e.g., ALDARA® cream). In the treatment of basal cell carcinoma (BCC), for example, standard dosing guidelines predict a severe reaction to ALDARA® in approximately 30% of patients according to historic published controls (see, e.g., the package insert for ALDARA® cream, incorporated by reference herein and available through the website for the U.S. Food and Drug Administration (“FDA”)).

Conditions in which imiquimod (e.g., ALDARA®) therapy is employed as a treatment means include malignant neoplasms (primary and metastatic) and benign neoplasms (e.g., neurofibroma, and warts), vascular malformations (e.g., port wine stains, hemangiomas), infections (e.g., parasitic infections), hair loss (alopecia areata), and thickening of skin (e.g., scleroderma or plaque morphea). Topical imiquimod (e.g., ALDARA®) therapy may be used to treat actinic keratosis, actinic chelitis, superficial or nodular basal cell carcinoma, melanoma metastases, cutaneous metastases from any neoplasm (benign or malignant), external genital warts, plantar warts, cervical intraepithelial neoplasia, vulvar intraepithelial neoplasia. Topical imiquimod (e.g., ALDARA®) therapy may also be used in conjunction with laser treatment, including for tattoo removal, and for the treatment of infectious conditions, such as cutaneous leishmaniasis Imiquimod (e.g., ALDARA®) therapy may also be employed at remote sites, for example, by injection, to stimulate the immune system for patients with cancer (e.g., metastatic cancer) or any disease requiring immunostimulation (e.g., HIV, systemic inflammatory disease).

Another therapeutic compound, 5-fluorouracil (5-FU), can have adverse side effects. 5-FU is the topical chemotherapeutic agent most widely used for cutaneous tumors and has been used to treat precancerous actinic keratosis lesions. 5-FU interferes with DNA synthesis in actively dividing cells, thereby causing tumor cell death. Patients self-treat by applying a topical cream of 5-FU for 4-6 weeks. This, however, results in increasing erythema and superficial erosions at affected sites. While these sites typically heal without scarring once the desired inflammatory end point is reached, some patients can experience pruritus and irritation, and, therefore, require close follow-up during the course of treatment to monitor response to the 5-FU treatment.

For individuals with psoriasis, treatment of this condition can involve topical vitamin D analogs. These vitamin D analogs, however, can cause local adverse effects, such as burning and irritation.

For the treatment of atopic dermatitis, topical calcineurin inhibitors (e.g., pimecrolimus, tacrolimus) can be used. Adverse side effects associated with these compounds, though, include burning and itching, though these effects may go away after the first few days of treatment.

Premalignant neoplasms such as acitinic keratosis can be treated with 5-FU cream or imiquimod 5% cream, but as discussed above for 5-FU and with respect to ALDARA®, both these therapeutic compounds have adverse side effects. In the case of 5-FU for the treatment of actinic keratosis, side effects include soreness, a similar side effect seen with the use of imiquimod 5% cream.

To “reduce an adverse side effect” means to reduce, ameliorate, prevent, or eliminate an unwanted or unpleasant side effect associated with a therapeutic compound. A “side effect” is typically an effect of a therapeutic compound that is in addition to its intended effect. The therapeutic compound can be any compound used to treat a disease or physical condition in a subject (e.g., a mammal, such as a human, dog, cat, horse, cow, or pig; or a bird, such as a chicken), including, without limitation, compounds for the treatment of non-cancerous (benign), precancerous, and cancerous (malignant) conditions, as well as compounds for the treatment of viral-mediated growths or disorders, chronic infections, and immune-mediated disorders. Likewise, in addition to biological and chemotherapies, the inventive methods described herein can be used to reduce unwanted side effects associated with, e.g., photodynamic therapy. In some embodiments, the inventive methods described herein can be used to reduce adverse side effects associated with a combination of drugs.

As used herein, the terms “drug,” “agent,” and “compound,” either alone or together with “therapeutic,” encompass any composition of matter or mixture which provides some pharmacologic effect that can be demonstrated in-vivo and/or in vitro. This includes small molecules, nucleic acids, proteins, antibodies, vaccines, vitamins, and other beneficial agents. As used herein, the terms further include any physiologically or pharmacologically active substance that produces a localized or systemic effect in a patient.

In certain embodiments, neurotoxin, such as botulinum toxin, is administered prior to the therapeutic compound. In other embodiments, neurotoxin, such as botulinum toxin, is administered subsequent to the therapeutic compound. In yet other embodiments, neurotoxin, such as botulinum toxin, is administered at the same time as the therapeutic compound.

In certain embodiments, botulinum toxin injections may reduce or eliminate adverse side effects associated with a therapeutic compound.

In certain embodiments, neurotoxin, such as botulinum toxin, administration reduces adverse side effects associated with a therapeutic compound, such as local skin reactions in a treatment area with a topically administered therapeutic compound, such as imiquimod (e.g., ALDARA® cream). In certain embodiments, for example, where a therapeutic compound is topically applied to a subject (e.g., a mammal), adverse side effects that may be associated with application of the therapeutic compound at the target site (e.g., skin or mucosa of a mammal) may be reduced according to the methods described herein. Such adverse side effects that may be reduced include itching, burning, bleeding, blistering, stinging, pain, induration, tenderness, soreness, sensitivity, irritation, erythema, flaking, scaling, dryness, scabbing, crusting, edema, erosion, ulceration, weeping, exudates, rash, vesicles, papules, infection, erosion, excoriation, and/or hypo- or hyperpigmentation.

Adverse side effects of a therapeutic compound can include systemic side effects. In certain embodiments, adverse side effects that may be reduced according to the methods described herein include headache, influenza-like symptoms, myalgia, fatigue, fever, diarrhea, upper respiratory tract infection, sinusitis, eczema, back pain, atrial fibrillation, chest pain, bacterial infection, fungal infection, viral infection, dizziness, nausea, vomiting, urinary tract infection, rigors, alopecia, lymphadenopathy, squamous carcinoma, dyspepsia, coughing, anxiety, and/or pharyngitis.

As discussed above, the anaerobic, gram positive bacterium, Clostridium botulinum, produces a potent polypeptide neurotoxin, botulinum toxin, which may cause a neuro-paralysis in humans. The neuro-paralysis is commonly referred to as botulism. Clostridium botulinum bacterium is commonly found in soil and will grow in improperly sterilized food containers. Signs and symptoms of botulism normally occur in humans within 18 to 36 hours after consuming foods containing a culture of Clostridium botulinum. It is thought that the botulinum toxin can pass through the lining of the gut and effect the peripheral motor neurons. The symptoms of botulinism begin with difficulty walking, swallowing, and speaking and progress to paralysis of the respiratory muscles resulting in death.

The affinity of botulinum toxin for muscle is well-known. Because of the extremely high affinity of the toxin for muscle, small doses of toxin may be used to elicit an effect. Smaller doses will result in fewer dose-related side effects such as the inadvertent spread of toxin through the tissues to neighboring structures, and resistance to future botulinum injections. There will be limited spread of the toxin since the toxin rapidly binds to the neuromuscular junction at the injection site. In fact, previous studies have shown that botulinum neurotoxin A complex, when injected into musculature, spreads no further than about a 7-8 mm distance (Tang-Liu, et al. “Intramuscular injection of 125I-botulinum neurotoxin-complex versus 125I-botulinum-free neurotoxin: time course of tissue distribution,” Toxicon 42 (2003) 461-469). In certain embodiments of the invention, the doses utilized are FDA approved for use in other neuromuscular conditions that are treated with botulinum toxin.

“Botulinum neurotoxin” or “botulinum toxin” may mean a botulinum neurotoxin as either pure toxin or complex. The botulinum neurotoxin can be from any suitable source, including botulinum neurotoxin purified from Clostridium botulinum or botulinum neurotoxin that is recombinantly produced. In one embodiment, the botulinum neurotoxin may be botulinum neurotoxin serotype A, B, C₁, D, E, F or G. In another embodiment, the botulinum neurotoxin is serotype A or serotype B. In yet another embodiment, the botulinum neurotoxin is serotype A. In a further embodiment, the botulinum neurotoxin is a mixture of two or more botulinum neurotoxin serotypes. In yet another embodiment, the botulinum neurotoxin is genetically modified.

As used herein a “therapeutically effective amount” of neurotoxin, such as botulinum neurotoxin, refers to an amount that is sufficient to reduce one or more side effects associated with the administration of a therapeutic compound. Typically, the therapeutically effective amount of neurotoxin is sufficient to reduce one or more side effects of a therapeutic compound that are considered adverse.

The therapeutically effective amount of the botulinum neurotoxin administered according to a method of the disclosed invention may vary according to age, weight, height, sex, muscle mass, area of target region, number of application sites, skin thickness, responsiveness to therapy and other patient variables known to the attending physician. The amount may also depend on the solubility characteristics of the botulinum neurotoxin chosen. Methods for determining the appropriate dosage are generally determined on a case by case basis by the attending physician. Such determinations are routine to one of ordinary skill in the art (See for example, Harrison's Principles of Internal Medicine (1998), edited by Anthony Fauci et al., 14^{th} edition, published by McGraw Hill).

Botulinum neurotoxins for use according to the present invention may be stored in lyophilized, vacuum dried form in containers under vacuum pressure or as stable liquids. Prior to lyophilization, the botulinum toxin may be combined with pharmaceutically acceptable excipients, stabilizers and/or carriers, such as albumin. The lyophilized material may be reconstituted with saline or water to create a solution or composition containing the botulinum toxin to be administered to the patient.

Other preparations of botulinum toxin are as follows:
Type A (DYSPORT®): Powder for solution for injection. Uncolored Type I glass vial containing a sterile white lyophilized powder.
Type B toxin (MYOBLOC®): Botulinum toxin type B (MYOBLOC®) is commercially available as a clear, colorless to light yellow solution of the drug in sterile water for injection. Each vial of MYOBLOC® injection contains 5000 units/mL of botulinum toxin type B; each mL of the injection also contains 0.5 mg of human albumin (to minimize adsorption of the toxin to the glass vial), 2.7 mg of sodium succinate, and 5.8 mg of sodium chloride. The commercially available injection of botulinum toxin type B (MYOBLOC®) has a pH of approximately 5.6.

Although the composition may only contain a single type of neurotoxin, such as botulinum neurotoxin serotype A, as the active ingredient to suppress neurotransmission, other therapeutic compositions may include two or more types of neurotoxins. For example, a composition administered to a patient may include botulinum neurotoxin serotype A and botulinum neurotoxin serotype B. Administering a single composition containing two different neurotoxins may permit the effective concentration of each of the neurotoxins to be lower than if a single neurotoxin is administered to the patient while still achieving the desired therapeutic effects.

Typically, about 0.1 unit to about 50 units of a botulinum neurotoxin serotype A (such as BOTOX®) may be administered per site (e.g., by injection or topical application), per patient treatment session. For a botulinum neurotoxin serotype A such as DYSPORT®, about 0.2 units to about 125 units of the botulinum neurotoxin serotype A may be administered per injection site, per patient treatment session. For a botulinum neurotoxin serotype B such as MYOBLOC®, about 10 units to about 1500 units of the botulinum neurotoxin serotype B may be administered per injection site, per patient treatment session.

In one embodiment, for BOTOX®, about 0.1 unit to about 20 units may be administered; for DYSPORT®, about 0.2 units to about 100 units may be administered; and, for MYOBLOC®, about 40 units to about 1000 units may be administered per injection site, per treatment session.

In another embodiment, for BOTOX®, about 0.5 units to about 15 units may be administered; for DYSPORT®, about 1 unit to about 75 units may be administered; and for MYOBLOC®, about 100 units to about 750 units may be administered per injection site, per patient treatment session.

As discussed above, botulinum toxin is available from multiple sources, such as from Allergan, Inc. as BOTOX®, a botulinum toxin type A (BTX-A) formulation; DYSPORT®, another BTX-A preparation available in Europe from Ipsen, Ltd; and MYOBLOC® (or NEUROBLOC® in Europe), a botulinum toxin type B (BTX-B) preparation available from Solstice Neurosciences, LLC.

Botulinum for use in the present invention can also be made by known pharmaceutical techniques by, for example, dissolving pharmaceutically acceptable botulinum toxin in a pharmaceutically acceptable carrier useful for injection, such that the botulinum is dissolved to the desired strength or concentration. These preparations can be made fresh or pre-made. Other pharmaceutically acceptable ingredients, such as preservatives, can be added. These preparations are made by techniques known in the art.

The amount of botulinum toxin to use may vary. The maximum dosage of botulinum A to administer should typically not exceed 500 units per injection session. In some embodiments, 0.01-100 units of botulinum A should be used. In other embodiments, the dosage of botulinum A should be in the range of from about 1 unit to about 50 units. In yet other embodiments, the dosage of botulinum A should be in the range of from about 5 units to about 40 units.

It is known that an electric current can enhance the absorption of botulinum toxin into tissues. Black, et al., Cell Biol-1986 August; 103(2): 535-44; Hesse et al., 1: Neurosci Lett. 1995 Dec. 1; 201(1) 37-40; Hesse, et al., Clin. Rehabil. 1998 October; 12(5): 381-8. Accordingly, one embodiment of the present invention is to apply an electric current to or around the area to be treated. This should decrease the amount of botulinum toxin needed for effective results.

If a different neurotoxin is used, such as botulinum B, C₁, D, E, F or G, the dosage should conform to the above dosage for botulinum A. Conversions, known in the art, can be used to calculate these dosages.

In one embodiment, the neurotoxin may be delivered in multiple doses for each patient treatment session. In another embodiment the neurotoxin may be delivered in about 1 to about 10 doses, depending on patient variables. In yet another embodiment the total therapeutically effective dose administered (e.g., about 0.1 unit to about 50 units) is divided evenly amongst multiple injection sites.

The concentration of neurotoxin, such as botulinum toxin, employed will depend on the type of neurotoxin used and on the target location to which the toxin is applied.

As defined herein, the vicinity of a target location that is a neoplasm refers to a distance that is typically within 7 mm from the edge or periphery of the neoplasm. Thus, if botulinum toxin is administered outside or away from the vicinity of the neoplasm, the toxin is generally administered at a distance of at least 7 mm from the neoplasm. It is known in the art that even when administered at high doses (e.g., about 70 units of botulinum neurotoxin complex), the majority of the toxin remains within about 7-8 mm of the site of injection (Tang-Liu et al., Toxicon 42 (2003) 461-469).

To reduce one or more adverse side effects associated with a therapeutic compound, the neurotoxin can be applied to an area outside of and/or surrounding the affected tissue being treated with the therapeutic compound. This may be accomplished by, for example, injecting the neurotoxin into one or more discrete locations along the periphery of the affected tissue. In certain embodiments, for example, where the affected tissue is a neoplasm, the neurotoxin can be injected into the noncancerous area around the neoplasm by, for example, injecting neurotoxin into one or more locations outside the vicinity of the perimeter of the neoplasm. Moreover, the neurotoxin can further be injected into the proximal lymph nodes, the distal lymph nodes, the thymus and/or the spleen.

Some conditions, such as chronic fatigue, HIV and AIDS, are systemic and do not involve a single organ system or tissue. In that event, neurotoxin may be administered by injecting the thymus, spleen or bone marrow. The lymph nodes may also be injected.

For injecting an organ or a tissue, especially one which cannot be visualized, the needle may be guided into place using conventional techniques. These techniques include, but are not limited to, palpitation, ultra sound guidance, CAT scan guidance, and X-ray guidance.

In one embodiment of the invention, a neurotoxin, such as botulinum toxin, is administered to reduce adverse effects associated with an anti-cancer drug. The anti-cancer drug may be, but is not limited to, an alkylating agent, an antimetabolite, an anthracycline, mitoxantrone, a topoisomerase inhibitor, a mitotic inhibitor, a steroid, a differentiation agent, a hormone, or an immunotherapy agent. In another embodiment the anti-cancer drug may be a mitotic inhibitor, including but not limited to the taxanes, such as paclitaxel (Taxol®) and docetaxel (Taxotere®); epothilones, which include ixabepilone (Ixempra®); the vinca alkaloids, such as vinblastine (Velban®), vincristine (Oncovin®), and vinorelbine (Navelbine®); and estramustine (Emcyt®).

As used herein, the term “neoplasm” includes benign (non-cancerous), pre-cancerous, or cancerous (malignant) tumors. The phrase “neoplastic cells” includes benign (non-cancerous), pre-cancerous, or cancerous (malignant) cells originating from a neoplasm. The phrase “non-neoplastic cells” refers to normal, healthy cells not originating from a neoplasm. Non-neoplastic cells are non-pre-cancerous, non-cancerous, non-diseased cells.

Previously, it has been shown that the neurotoxin, botulinum toxin, can induce an increased inflammatory response in the tissue surrounding a tumor (see U.S. Pat. No. 8,343,929, incorporated by reference herein). In certain embodiments, the methods described herein also positively modulate the immune system to enhance cellular or humoral mechanisms.

A review of relevant anatomy follows:

Localization of Lymphatic Tissue

Besides blood vessels, the human body has a system of channels that collects fluid from the tissue spaces and returns it to the blood. This fluid is called lymph, and in contrast to blood, it circulates in only one direction, toward the heart.

The lymphatic capillaries originate as blind-ended, thin walled vessels. They are comprised of thin walled endothelium. These thin walled vessels ultimately converge and end up as two main trunks, the thoracic duct and the right lymphatic duct. These enter into the junction of the left internal jugular vein and the left subclavian vein, and into the confluence of the right subclavian vein and the right internal jugular vein. Interposed in the path of the lymphatic vessels are lymph nodes. The larger lymphatic vessels have a smooth muscle layer that helps propel lymph flow through the channels and unidirectional lymph flow occurs secondary to the presence of many one-way valves.

The lymphatic ducts of large size (thoracic and right lymphatic ducts) have a reinforced smooth muscle layer in the middle, in which the muscles are oriented longitudinally and circularly. They contain vasa vasorum and a rich neural network (Junqueira L, Basic Histology, 1986, Lange Medical Publications, page 269).

Lymphoid Tissue

The spleen, thymus and bone marrow are also considered lymphoid tissue. These lymphoid organs are classified as either being central or peripheral and encapsulated (e.g. spleen or lymph nodes) or unencapsulated (e.g. tonsils, peyers patches in the intestine, lymphoid nodules found throughout the mucosa of the alimentary, respiratory, urinary and reproductive tract). (Junqueira L, Basic Histology, 1986, Lange Medical Publications, page 269)

In general, lymphoid cells begin in a “central” lymphoid organ where lymphoid precursors undergo antigen-independent proliferation and acquire surface antigens that mark them as committed to either the cellular or humoral immune response. The thymus is the central organ where lymphocytes take on the capacity to participate in the cellular immune response (T cells). Cells migrate through the blood from the bone marrow to the thymus, where they proliferate, giving rise to T cells. These lymphocytes are responsible for cell-mediated immune reactions. The bone marrow is where progenitor cells differentiate into humoral immune cells (B-cells) which ultimately become plasma cells and secrete immunoglobulins and provide the humoral immune response. Lymphocytes leave the central lymphoid organs and populate specific regions of “peripheral” lymphoid organs, such as lymph nodes, spleen, peyer's patchs and diffuse unencapsulated lymphoid tissue in the mucosa of the digestive, respiratory, urinary and reproductive tracts (Junqueira L, Basic Histology, 1986, Lange Medical Publications, page 269).

Spleen: The spleen is the largest lymphatic organ in the circulatory system. The spleen is a site of formation of activated lymphocytes. It serves to filter and modify the blood.

Thymus: The thymus is a central lymphoid organ located in the mediastinum. There is intense lymphocytic proliferation that occurs in the thymus during embryonic through pre-pubertal development. This is where cells proliferate that become T lymphocytes, the cells responsible for cell-mediated immunity. From the thymus, these T cells leave through blood vessels to populate the peripheral lymphoid organs, especially lymph nodes and the spleen.

Bone Marrow: The bone marrow is also a central organ, but it gives rise to B cells, which ultimately differentiate into plasma cells and secrete antibodies (the humoral immune system). After differentiation, the B cells travel to lymph nodes, the spleen and especially Peyer's patches in the intestine (Junqueira, supra, page 312).

Lymph Nodes: Lymph nodes are encapsulated areas of peripheral lymphoid tissue. They are distributed throughout the body, always along the course of lymphoid vessels, which carry lymph into the thoracic and lymphatic ducts (Junqueira, supra, page 313). Lymph nodes are aggregated in particular sites such as the neck, axillae, groins and para-aortic region. The precise location of lymph nodes is well-known. See, e.g., Le, UAMS Department of Anatomy—Lymphatics Tables (Jul. 16, 2005), which is incorporated herein by reference in its entirety.

Lymph enters the lymph nodes through the afferent lymphatic channel and exits through the efferent channel. Flow is unidirectional. As lymph flows through the sinuses, 99% or more of the antigens or other debris are removed by the phagocytic activity of the macrophages within the node. Some of the material is trapped on the surface of dendritic cells, which is then exposed on the surface of the dendritic cell and recognized and acted upon by immunocompetent lymphocytes. The parenchyma of a lymph node has three general regions, the cortex, paracortex and medulla.

In the cortex, if a B cell recognizes an antigen (and sometimes with the help of T cells) the B cell may become activated and synthesize antibodies which are released into the lymph fluid then into the circulation. Activated B cells remain within the lymph node. Unstimulated B cells pass out of the lymph node and return to the general circulation.

T cells remain predominantly in the paracortex region of the lymph node. Activated T cells pass into the circulation to reach the peripheral site. Other cell types, predominantly antigen presenting cells, reside in the paracortical region of the lymph node.

The medulla is rich in plasma cells which produce further antibodies, and macrophages.

Unencapsulated tissue: Unencapsulated lymphoid tissue can be found mainly in the loose connective tissue of many organs, mainly in the lamina propria of the digestive tract, upper respiratory tract and urinary passages (Junqueira, supra, page 323). The palatine, lingual and pharyngeal tonsils are another main site of unencapsulated lymphoid tissue. This so-called mucosa-associated lymphoid tissue (MALT) includes gut-associated lymphoid tissue (GALT), bronchial/tracheal-associated lymphoid tissue (BALT), nose-associated lymphoid tissue (NALT), and vulvovaginal-associated lymphoid tissue (VALT). Additional MALT exists within the accessory organs of the digestive tract, predominantly the parotid gland.

MALT may comprise a collection of lymphoid cells or may include small solitary lymph nodes. Stimulation of B lymphocytes leads to the production of immunoglobulin A (IgA) and IgM within the peyers patches. Additionally, epithelial surfaces contain M cells which are specialized cells that absorb, transport and present antigens to subepithelial lymphoid cells, such as CD4 type 1 helper cells, antigen presenting cells and memory cells.

Generally, lymphocytes contain antigen receptors that trigger differentiation. In peripheral organs, lymphocytes interact with appropriate antigens, enlarge then divide. Some become effector cells, and others become memory cells that are responsible for the secondary immune response. To generate an immune response and for effector cells to be generated, antigen must be delivered to them. This is the job of antigen presenting cells which include dendritic cells, macrophages and Langhans cells in the epidermis.

Effector cells can be activated B- or T-cells. B-cell effector cells are plasma cells that secrete immunoglobilins into the surrounding connective tissues. T-cell effector cells are of several types and include helper T cells, suppressor T cells and cytotoxic T cells. Cells attacked include tumor and viral-infected cells. T cells and macrophages secrete lymphokines that regulate the proliferation of both B and T cells.

Lymphatic Flow

The lymphatic system is found in almost all organs except the central nervous system and the bone marrow. The lymphatic circulation is aided by the action of external forces such as the contraction of surrounding skeletal muscle on their walls. (Junqueira, supra, page 269). These forces cause transportation along lymphatic channels. Contraction of smooth muscle in the walls of the larger lymphatic vessels also helps propel lymph. The transport of lymph depends on active and passive driving forces. The active driving force resulting from intrinsic pump activity in some lymph vessels plays an important role in the propulsion of lymph flow (Hosaka K, et al. Am J Physiol Heart Circ Physiol 284, 2003, abstract) There is myogenic tone in lymph channels. It has been demonstrated that the Rho kinase pathway (which is inhibited by botulinum toxin) helps regulate the lymph pump activity (Hosaka, supra). In fact, it has been demonstrated that lymph vessels are capable of regulating flow through intrinsic mechanisms (Ferguson M K, et al. Lymphology 27(2), 1994 abstract and, Muthuchamy M, et al. Molecular and Functional analyses of the contractile apparatus in lymphatic muscle. FASEB J 17, 2003, abstract). Larger lymphatic ducts contain smooth muscle and a rich neural network (Junqueira, supra, page 269).

Several factors aid the flow of lymph fluid from tissue spaces to lymph nodes and finally to the venous bloodstream: 1) “Filtration pressure” in tissue spaces, generated by filtration of fluid under pressure from the haemal capillaries; 2) Contraction of neighboring muscles compresses the lymph vessels, moving lymph in the direction determined by the arrangement of valves; 3) Pulsation of adjacent arteries; 4) Respiratory movements and the low blood pressure in the brachiocephalic vein during inspiration; 5) Smooth muscle in the walls of lymphatic trunks is most marked proximal to their valves. Pulsatile contractions in the thoracic duct are known to occur also.

Botulinum Toxin Will Weaken Lymphatic Transit

The effect of botulinum toxin on skeletal muscle is well-known. In fact, it is the basis of therapy for conditions such as strabismus, dystonias and other spastic muscle conditions. The FDA has granted approval of botulinum therapy for strabismus, blepharospasm, cervical dystonias and others. The range of doses needed to paralyze various muscles in the body is well-established.

A regional injection of botulinum toxin around a cancer or other diseased tissue will exploit the well-known binding affinity of botulinum for muscle. Skeletal muscle, smooth muscle, lymphatic muscle, blood vessel muscle and pericyte muscle are non-limiting targets of the methods of the instant invention. The paralysis of surrounding skeletal or smooth muscle may limit the contractile extrinsic forces on lymphatic structures that normally facilitate flow of lymph through lymphatic channels. The intrinsic muscles within lymphatic tubules may be paralyzed or weakened by botulinum therapy. The smooth muscle wall of blood vessels may be weakened as well.

Immune responses can be innate (natural) or acquired (adaptive). Innate immunity is mediated by cells or soluble factors which naturally exist in tissues of body fluids and can interfere with tumor growth (Whiteside T L. J. Allergy Clin Immunol 2003; 111, S677-86). The hematopoietic cells included are macrophages, granulocytes, natural killer cells, non-MHC-restricted T cells and gamma/delta T cells. Also, natural antibodies directed at the surface components of tumor cells, complement components, C reactive protein, serum amyloid protein, mannose-binding protein are also included (Whiteside, supra). Adaptive immunity is mediated by T cells which recognize tumor-derived peptides bound to self-MHC molecules expressed on antigen presenting cells (APC). These cells include cytolytic effector cells, which are CD8+ and MHC class I restricted, but also helper CD4+ T cells (Whiteside, supra).

The invention will now be further described by way of the following non-limiting examples.

### EXAMPLES

The following non-limiting examples demonstrate the ability of botulinum toxin to reduce adverse side effects associated with the administration of a therapeutic compound:

### Example 1

### Co-Administration of Botulinum Toxin (BOTOX®) with ALDARA® Cream Therapy Reduces the Incidence of Complications to ALDARA® Cream

Regarding safety in this study design, the target organ is an FDA-approved site for botulinum toxin in conditions such as hyperhidrosis or glabellar lines. A small dose was applied to further maximize safety in this study. As a reference, it is not unusual to inject 100 units of botulinum toxin at one session into the skin for patients being treated for palmar hyperhidrosis. This study used 10 units only.

### Criteria for Subject Selection

### NUMBER OF SUBJECTS: 10

GENDER OF SUBJECTS: Men and women

### AGE OF SUBJECTS: 18-90

RACIAL AND ETHNIC ORIGIN: No enrollment restrictions

### Inclusion Criteria:

1) Have at least one superficial or nodular basal cell carcinoma
2) Minimum area of tumor of 0.5 cm²
EXCLUSION CRITERIA: People with known hypersensitivity to botulinum toxin
VULNERABLE SUBJECTS: Not enrolled in this study

### Methods and Procedures

A total of 10 patients with biopsy-proven superficial basal cell carcinoma or nodular basal cell carcinoma were eligible for this pilot study. Patients with suspicious basal cell lesions were informed at the time of biopsy that if the diagnosis of basal cell carcinoma was confirmed, topical immunotherapy was the treatment of choice and that they may enter a clinical trial using botulinum toxin injections to enhance efficacy of treatment.

One to two weeks after the diagnosis was confirmed, 10 units of type A botulinum toxin (BOTOX®, Allergan Inc., Irvine, Calif.) was injected around the non-cancerous area of the original lesion. Approximately 5 separate injections of 2 units each were injected into the dermis, into the noncancerous area surrounding the neoplasm. The 5 injections of 2 units each were injected 1 cm away from the neoplasm along the noncancerous perimeter surrounding the neoplasm.

Approximately 3-5 days after the botulinum toxin was injected, each patient began standard treatment with topical 5% ALDARA® cream. ALDARA® was applied five times per week for a total of six weeks prior to normal sleeping hours. In addition to the tumor itself, a 1 cm area of normal skin around the lesion was treated with ALDARA®. Four weeks after the ALDARA® treatment in conjunction with botulinum toxin was completed, a repeat biopsy was performed to assess for the presence of cancer.

The following parameters were monitored: Patient diary, photographs of lesion, physician observation, and overall response rate to treatment.

### Patient Monitoring

Patient diary: Diary information was entered just prior to botulinum toxin injection, and then on a weekly basis until the time of the follow-up biopsy. The patient recorded the following symptoms on a scale of 1-5, with 5 being severe:

Pain at site

Blistering at site

Redness at site

Sensitivity at site

### Physician Monitoring

Patients were seen in the physician's office every 2 weeks for the first 6 weeks, then 4 weeks later for repeat shave biopsy. Photographs, physician observation, and response rate were monitored and recorded.

Photographs:

Suspicious lesions were photographed prior to diagnostic shave biopsy. Lesions were then photographed every 2 weeks until follow-up biopsy.

Physician Score:

The physician score was recorded on a scale of 1-5, with 5 being severe:

Blistering at site

Redness at site

Sensitivity at site

Overall Response Rate:

Response rate was recorded at the time of follow-up biopsy, at approximately 10 weeks after the start of treatment.

### Adverse Reactions and Stopping Rules

The principal investigator monitored patients and data.

Adverse reactions could occur immediately after injection of botulinum toxin but before therapy with ALDARA® cream (toxin-related reactions), or after patient begins therapy with ALDARA® cream (combination of toxin plus ALDARA® reaction). Adverse reactions were considered either minor or major.

Toxin-related reactions: Hypersensitivity reactions to botulinum toxin were monitored.

Toxin plus ALDARA® cream reactions: Minor reactions included pain, redness or blistering at the site of injection scaled 1/5, 2/5, or 3/5. Major reactions included a score of 4/5 or 5/5. If four consecutive patients developed a major reaction in more than 3 consecutive physician recordings, the study was to be terminated.

### Risk Assessment

RISK CATEGORY: Greater than Minimal.

### Potential Risk:

It was believed that the risk of the study was very small. The target organ was already an FDA approved site of injection/therapy, and the dose of toxin being used in this study was well below the FDA recommended dose for other cutaneous/subcutaneous conditions. Regardless, the potential for complications to botulinum toxin injections was monitored.

### Results

10 patients were enrolled in the study: 8 with superficial basal cell carcinoma (sBCC) and 2 with nodular basal cell carcinoma (nBCC).

No botulinum toxin-related reactions were observed in the patients.

Application of ALDARA® cream to each patient followed standard dosing guidelines (see, e.g., the package insert for ALDARA® cream, available through the FDA website). Standard dosing guidelines predict a severe reaction to ALDARA® in approximately 30% of patients according to historic published controls, and an efficacy rate in sBCC of approximately 80-87% (see, e.g., the package insert for ALDARA® cream, available through the FDA website).

### 1) Side Effect Reduction:

Of the 10 patients enrolled, 1 was lost to follow-up (See Table 1, Patient #8). Of the 9 patients analyzed, 0 had a severe reaction, 1 had a moderate reaction, and 8 had a mild reaction to ALDARA® cream. This reduction of severe side effects to ALDARA® is statistically significant (p=0.0404) when compared to the historic severe reaction rate of 30%. The test p value was calculated based on 1-sided exact binomial proportion test.

2) Efficacy of Treating sBCC:

Of the 8 patients with sBCC, 1 could not be included in analysis on efficacy (one patient did not properly follow ALDARA® protocol (See Table 1, Patient #8)). 7 patients were therefore analyzed with regard to efficacy of treatment, and all 7 had negative follow-up biopsies, resulting in a 100% clearance rate.

In addition, in patient 7, two lesions were both treated with ALDARA®, but only one of these lesions was also treated with peritumoral botulinum toxin (see Table 1). The lesion treated with peritumoral botulinum toxin in addition to ALDARA® had only a mild reaction to ALDARA® (see Table 1, Patient #7), but the lesion treated with only ALDARA® had a moderate reaction.

The results of the study are presented in Table 1. As indicated in Table 1, a one-time, long-acting injection of botulinum toxin into a non-cancerous area around a basal cell carcinoma reduced the incidence of complications to ALDARA® cream.

The results of botulinum toxin co-administration with ALDARA® cream for the treatment of basal cell carcinoma demonstrate significantly increased tolerability of ALDARA® therapy, potentially better scarring, fewer treatment breaks secondary to local side effects, and the ability to treat regions that are not typically amenable to ALDARA® therapy, such as the face.

TABLE 1

| | Patient # | | | | | |
|---|---|---|---|---|---|---|
| | 1 | 2 | 3 | 4 | 5 | 6 |
| Location/ | Left | Left | Left cheek- | crown of | Right upper | Right cheek- |
| Type of BCC | forehead- | lateral | nBCC | scalp- | abdomen- | sBCC |
| | sBCC | shin- | | nBCC | sBCC | |
| | | sBCC | | | | |
| Summary | Mild | Mild | Mild | Mild to | Mild | Mild |
| reaction to | | | | moderate | | |
| ALDARA ® | | | | | | |
| and | | | | | | |
| botulinum | | | | | | |
| toxin: | | | | | | |
| PHYSICIAN | | | | | | |
| ASSESSMENT | | | | | | |
| Final biopsy | PAPILLARY | INFLAMED | Residual BCC, | Patient had | INFLAMED | DENSE |
| results status | DERMAL | SCAR; no | at least | MOHS | SUPERFICIAL | LICHENOID |
| post | SCAR AND | residual | superficial | surgical | DERMAL | INFLAMMATION |
| ALDARA ® | SURFACE | BCC is | type, extending | procedure | SCAR. | WITH |
| and | OF | identified. | to the tissue | performed. | Multiple levels | MELANOPHAGES, |
| botulinum | INFLAMED | | edge; SCAR | | fail to reveal | MILD DERMAL |
| toxin | ADNEXAL | | AND | | BCC. | FIBROSIS, AND |
| treatment | STRUCTURES | | INFLAMMATION. | | | FOCAL |
| | residual BCC | | | | | SUPERFICIAL |
| | is not | | | | | PUSTULAR |
| | identified. | | | | | FOLLICULITIS |
| | | | | | | Multiple levels fail |
| | | | | | | to reveal BCC. |

| | Patient # | | | |
|---|---|---|---|---|
| | 7 | 8 | 9 | 10 |
| Location/ | Left lateral | Left upper | Right anterior | Right |
| Type of BCC | midback and | back- | thigh- | upper |
| | Left upper | sBCC | sBCC | chest- |
| | arm- | | | sBCC |
| | sBCC | | | |
| Summary | Mild | — | Mild | Mild |
| reaction to | | | | |
| ALDARA ® | | | | |
| and | | | | |
| botulinum | | | | |
| toxin: | | | | |
| PHYSICIAN | | | | |
| ASSESSMENT | | | | |
| Final biopsy | ARM | No biopsy | SCAR | No |
| results status | (ALDARA ® | | CONSISTENT | evidence of |
| post | AND | | WITH | BCC |
| ALDARA ® | BOTULINUM | | ALDARA ® | |
| and | TOXIN | | TREATMENT; | |
| botulinum | TREATMENT)- | | no evidence of | |
| toxin | SURFACE | | BCC. | |
| treatment | OF SCAR; no | | | |
| | evidence | | | |
| | of BCC. | | | |
| | BACK | | | |
| | (ALDARA ® | | | |
| | TREATMENT | | | |
| | ONLY)- | | | |
| | SURFACE OF | | | |
| | SCAR, | | | |
| | INFLAMED; | | | |
| | no evidence of | | | |
| | BCC. | | | |

### Example 2

### Co-Administration of Botulinum Toxin (MYOBLOC®) with ALDARA® Cream Therapy for sBCC

### Methods and Procedures

A total of 3 patients with biopsy-proven superficial basal cell carcinoma (sBCC) were treated in this pilot study. Type B toxin (MYOBLOC®) was injected into the non-cancerous area around the original lesion. Approximately 5 separate injections were injected into the dermis, into the noncancerous area surrounding the neoplasm.

The dose of toxin was determined according to the approximate area of the cancerous lesion and using a ratio of 1:75 type A:B, the dose of type B toxin injected was 750 units of type B toxin per cm².

Approximately 3-5 days after the botulinum toxin was injected, each patient began standard treatment with topical 5% ALDARA® cream. ALDARA® was applied five times per week for a total of six weeks prior to normal sleeping hours. In addition to the tumor itself, a 1 cm area of normal skin around the lesion was treated.

Four weeks after the ALDARA® is completed, a repeat biopsy is performed to assess for the presence of cancer.

### Results

3 superficial BCCs treated with MYOBLOC®/ALDARA®:

1) 69 year old patient: location of sBCC—shin

2) 48 year old patient: location of sBCC—shin

3) 53 year old patient: location of sBCC—forehead

All 3 patients had no MYOBLOC® application reaction, and all 3 patients had a mild reaction to ALDARA®.

### Example 3

### Co-Administration of Botulinum Toxin (MYOBLOC®) with ALDARA® Cream them for Actinic Keratosis

### Methods and Procedures

A total of 6 patients with at least two separately identifiable, clinically diagnosed actinic keratosis were treated in this study.

ALDARA® cream is indicated for the topical treatment of clinically typical, nonhyperkeratotic, nonhypertrophic actinic keratoses on the face or scalp in immunocompetent adults. ALDARA® 5% cream is typically applied 2 times per week for a full 16 weeks.

On the day of treatment, the two lesions previously identified were treated. The region around one lesion was injected with MYOBLOC® and the other with saline. The patient was blinded to treatment. Type B toxin (MYOBLOC®) was injected into the surrounding normal area around the original lesion. Approximately 5 separate injections were injected into the dermis, into the normal area surrounding the lesion. The other lesion was injected with the same number of injections and the same volume of saline.

The dose of toxin was determined according to the approximate area of the actinic keratosis receiving the MYOBLOC®. Using a ratio of 1:75 type A:B, the dose of type B toxin injected was 750 units of type B toxin per cm². Maximum dose to be administered was 1500 units of type B toxin (total 2 cm²).

Approximately 2 weeks after the botulinum toxin was injected, each patient began standard treatment with topical 5% ALDARA® cream. ALDARA® was applied two times per week for a total of sixteen weeks prior to normal sleeping hours.

### Results

6 patients with actinic keratosis were treated with MYOBLOC®/ALDARA®:

1) 52 year old patient: location of actinic keratosis—scalp

2) 52 year old patient: location of actinic keratosis—forehead

3) 56 year old patient: location of actinic keratosis—scalp

4) 62 year old patient: location of actinic keratosis—arm

5) 58 year old patient: location of actinic keratosis—forehead

6) 53 year old patient: location of actinic keratosis—temple

All 6 patients had mild/none reaction at MYOBLOC®/ALDARA® site whereas the control lesion had mild/moderate reaction.

Having thus described in detail embodiments of the present invention, it is to be understood that the invention defined by the above paragraphs is not to be limited to particular details set forth in the above description as many apparent variations thereof are possible without departing from the spirit or scope of the present invention.

Each patent, patent application, and publication cited or described in the present application is hereby incorporated by reference in its entirety as if each individual patent, patent application, or publication was specifically and individually indicated to be incorporated by reference.

What is claimed is:

1. A method of reducing one or more side effects associated with a therapeutic compound in a subject, comprising administering to the subject a therapeutically effective amount of neurotoxin in combination with the therapeutic compound, wherein the therapeutically effective amount of neurotoxin reduces one or more side effects of the therapeutic compound.

2. The method of claim 1, wherein the neurotoxin is administered before the therapeutic compound is administered.

3. The method of claim 1, wherein the neurotoxin is administered at the same time as the therapeutic compound.

4. The method of claim 1, wherein the neurotoxin is administered after the therapeutic compound.

5. The method of claim 1, wherein the therapeutic compound is selected from the group consisting of an alkylating agent, an antimetabolite, an anthracycline, mitoxantrone, a topoisomerase inhibitor, a mitotic inhibitor, a steroid, a differentiation agent, a hormone, and an immunotherapy agent.

6. The method of claim 5, wherein the therapeutic compound is imiquimod.

7. The method of claim 6, wherein the imiquimod is administered topically.

8. The method of claim 1, wherein the neurotoxin is botulinum neurotoxin.

9. The method of claim 8, wherein the therapeutic compound is administered topically.

10. The method of claim 1, wherein the one or more side effects are adverse side effects at or near the site of administration of the therapeutic compound and wherein the adverse side effects are selected from the group consisting of: pain, erythema, soreness, swelling, blistering, and sensitivity.

11. The method of claim 10, wherein the therapeutic compound is imiquimod.

12. The method of claim 11, wherein the imiquimod is administered topically.

13. The method of claim 12, wherein the site of topical administration of imiquimod is to a neoplasm, an actinic keratosis, or a genital wart.

14. The method of claim 13, wherein the neoplasm is a basal cell carcinoma.

15. The method of claim 14, wherein the basal cell carcinoma is superficial or nodular.

16. The method of claim 8, wherein the botulinum neurotoxin is botulinum neurotoxin type A.

17. The method of claim 8, wherein the botulinum neurotoxin is botulinum neurotoxin type B.

18. The method of claim 8, wherein the dose of botulinum neurotoxin does not exceed 500 units per application.

19. The method of claim 18, wherein the dose of botulinum neurotoxin is between about 0.01 to about 100 units per application.

20. The method of claim 19, wherein the dose of botulinum neurotoxin is between about 1 unit to about 50 units per application.

21. The method of claim 8, wherein the botulinum neurotoxin is applied topically or by injection.

22. The method of claim 21, wherein the botulinum neurotoxin is applied by injection.

23. The method of claim 13, wherein the neurotoxin is botulinum neurotoxin, and wherein the botulinum neurotoxin is applied to the non-cancerous area around the neoplasm.

24. The method of claim 23, wherein the side effects of the imiquimod are reduced such that they are mild or do not occur.
